(19) United States
(12) Patent Application Publication
(10) Pub. No.: US 2011/0048446 A1
(43) Pub. Date: Mar. 3, 2011

(54) Method of Delivering a Hair-Care Benefit and a Fragrance

(76) Inventors: Peter Marte Torgerson, Washington Court House, OH (US); Barbara Kay Hendricks, West Chester, OH (US)

(21) Appl. No.: 12/776,505

(22) Filed: May 10, 2010

Related U.S. Application Data
(60) Provisional application No. 61/177,437, filed on May 12, 2009.

(51) Int. Cl. A61K 8/18

(52) U.S. Cl. 132/202

(57) ABSTRACT

The present invention relates to a method of providing a benefit to the hair comprising applying a hair-care composition to the hair and applying a moist-heat wrap to the hair. The moist-heat wrap comprises a suppressed fragrance releaseably associated with a suppressing agent. The benefit is selected from the group consisting of hair conditioning, hair shine, smooth hair, soft hair, healthy hair, hair strand alignment, dandruff-free hair, hair coloring, and combinations thereof.

### CROSS REFERENCE TO RELATED APPLICATION

This application claims the benefit of U.S. Provisional application Ser. No. 61/177,437 filed May 12, 2009.

### FIELD OF THE INVENTION

The present invention relates to methods of delivering a hair-care benefit in combination with a fragrance by the use of a moist-heat wrap comprising a suppressed fragrance.

### BACKGROUND OF THE INVENTION

A variety of approaches have been developed to deliver hair-care actives to the hair. A common method of providing a hair-care active is through the use of hair-care compositions containing actives such as actives to deliver hair conditioning, hair shine, smooth hair, soft hair, healthy hair, hair strand alignment, dandruff free hair, and combinations thereof. Additionally, traditional methods of conditioning hair combine the use of hair conditioners with heat such as a hot towel or a hair dryer. However, these heat treatments are either difficult and inconvenient to use, or do not provide heat of a consistent temperature that is safe to use on the hair for a length of time sufficient to deliver a hair-care benefit. Additionally, use of a hair dryer cannot provide a moist heat to the hair.

Disposable heat wraps have become a popular way of applying heat to relieve discomfort of temporary or chronic body aches and pains. Disposable heat wraps typically comprise an exothermic composition for generating heat, wherein the exothermic composition typically comprises metal powder, salts, and water that allows the exothermic composition to release heat upon oxidation of the metal powder. Other disposable or reusable devices can use energy produced by neutralization of acids and bases; heat of hydration of inorganic salts; re-heatable gels; and electrical energy to produce heat. Such devices usually produce heat but contain little moisture.

Recently, moist heat sources have been developed which are easy and convenient to use with a hair-care active, and which also provide consistent temperature to the hair for a length of time sufficient to deliver a benefit to the hair. However, the experience of using these moist-heat wraps on the hair is far from glamorous. There is a desire for an at-home spa-like experience while using a moist-heat wrap. Accordingly, there is a need for a scented moist-heat wrap. There is also a desire to provide a moist-heat wrap that is able to differ in perceived scent over time—for example, to make the experience of using the wrap luxurious or as a mechanism of signaling an amount of time has passed.

None of the existing art provides all of the advantages and benefits of the present invention.

### SUMMARY OF THE INVENTION

A hair-care benefit may be achieved by incorporating one or more benefit-delivery agents into a moist-heat wrap. The addition of a fragrance to the moist-heat wrap may engender a spa-like or other enjoyable consumer experience. In one embodiment, the moist-heat wrap may deliver a first fragrance, perceivable upon activation/removal of the heat wrap from its packaging. In lieu of or in addition to the first fragrance, the moist-heat wrap may deliver a second fragrance, perceivable after an amount of time after activation/removal of the heat wrap from its packaging. So, in some embodiments, the moist-heat wrap may differ in perceived scent over time. In further embodiments, the moist-heat wrap may deliver more than two fragrances.

In particular, the present invention is directed methods of delivering a hair-care benefit and a suppressed fragrance by the use of a steam-generating unit.

In one embodiment, a method of providing a benefit to the hair comprises (a) applying a hair-care composition to the hair; and (b) applying a moist-heat wrap to the hair, wherein the moist-heat wrap comprises a suppressed fragrance releaseably associated with a suppressing agent; wherein the benefit is selected from the group consisting of hair conditioning, hair shine, smooth hair, soft hair, healthy hair, hair strand alignment, dandruff-free hair, hair coloring, and combinations thereof. In certain embodiments, the suppressing agent is cyclodextrin.

### BRIEF DESCRIPTION OF THE DRAWINGS

While the specification concludes with claims particularly pointing out and distinctly claiming the present invention, it is believed the same will be better understood from the following description taken in conjunction with the accompanying drawings in which:

FIG. 1 is a top view of a moist-heat wrap of the present invention.

FIG. 2 is a cross section view of the moist-heat wrap of FIG. 1.

### DETAILED DESCRIPTION OF THE INVENTION

All percentages, parts, and ratios are by weight, unless otherwise specified. The number of significant digits conveys neither limitation on the indicated amounts nor on the accuracy of the measurements. All measurements, unless otherwise provided, are understood to be made at about 25° C. and at ambient conditions, where “ambient conditions” means conditions under about one atmosphere of pressure and at about 50% relative humidity. All ranges are inclusive and combinable; therefore, every range given throughout this specification will include every narrower range that falls within such broader range as if such narrower ranges were all expressly written herein.

“Fragrance,” as used herein, refers to formulations or compositions made up of one or a mixture of perfume chemicals/raw materials and/or odor-controlling materials. Differences between fragrances may result from same chemical make-ups (presence and concentration of individual perfume chemicals/raw materials and/or odor-controlling materials) but different overall concentrations, same overall concentrations but different chemical make-ups, different chemical make-ups and different overall concentrations, and the like.

### I. BENEFIT DELIVERY

In one aspect, the present invention relates to a method of delivering a hair-care benefit such as conditioning, hair shine, smooth hair, soft hair, healthy hair, hair strand alignment, or dandruff free hair by the use of a hair-care composition, with the use of a moist-heat wrap of the present invention. It is believed that moist heat delivered by a steam-generating unit acts to swell the hair thus enhancing the penetration of the beneficial hair actives. It is also believed that moist heat delivered by the steam-generating unit may affect the ability of materials on the hair surface, particularly the high molecular weight lubricants, such as silicone, to reduce friction, by affecting their distribution on the hair surface. In another aspect, the present invention relates to a method of delivering a skin-care benefit such as moisturizing, exfoliating, or hair removal via a moist-heat wrap.

In some embodiments, a composition may be applied directly to the skin and/or hair before or after use of the moist-heat wrap of the present invention in order to achieve a benefit. In other embodiments, the moist-heat wrap of the present invention may incorporate a composition to be delivered to the skin and/or hair. The composition may be incorporated into the moist-heat wrap integrally throughout at least a portion of the wrap, or it may be a layer or coating on the surface of the moist-heat wrap which comes into contact with the hair or skin. The moist-heat wrap may comprise a composition either integral with the contact surface or as a layer on top of the contact surface.

The compositions of the present invention can be in the form of rinse-off products or leave-on products, and can be formulated in a wide variety of product forms, including but not limited to creams, gels, emulsions, mousses and sprays. Additionally, the hair-care compositions of the present invention can be a conditioner, a shampoo, a styling product, an anti-dandruff product and/or any combination thereof.

The composition of the present invention may be an emulsion, having viscosity at 25° C. of at least about 5,000 cP preferably from about 8,000 cP to about 50,000 cP, more preferably from about 15,000 cP to about 35,000 cP. Viscosity is determined by a Brookfield RVT, at 20 RPM. The compositions of the present invention preferably have a pH of from about 2.5 to about 7, more preferably from about 3 to about 6.8, most preferably from about 3.5 to about 6.5 Higher pH can be utilized as long as the composition retains a viscosity of at least about 8,000 cP at 25° C. In various embodiments, the composition may comprise one or more of the following components:

A. Nonvolatile, Low Melting Point Fatty Alcohol

The compositions of the present invention may comprise from about 0.1% to about 10%, by weight, preferably from about 0.1% to about 5%, more preferably from about 0.25% to about 1%, of a nonvolatile, monohydric, low melting point fatty alcohol. By nonvolatile what is meant is they have a boiling point at 1.0 atmospheres of at least about 260° C., preferably at least about 275° C., more preferably at least about 300° C. The fatty alcohols hereof have a melting point of 30° C. or less, preferably about 25° C. or less, more preferably about 22° C. or less.

Suitable fatty alcohols include unsaturated straight chain fatty alcohols, saturated branched chain fatty alcohols, saturated C8-C12 straight chain fatty alcohols, and mixtures thereof. The unsaturated straight chain fatty alcohols will typically have one degree of unsaturation. Especially preferred unsaturated straight chain fatty alcohols include oleyl alcohol and palmitoleic alcohol. Di- and tri-unsaturated alkenyl chains may be present at low levels, preferably less than about 5% by total weight of the unsaturated straight chain fatty alcohol, more preferably less than about 2%, most preferably less than about 1%. Exemplary branched chain alcohols for use herein include isostearyl alcohol, octyl dodecanol, and octyl decanol. Examples of saturated C8-C12 straight chain alcohols include octyl alcohol, caprylic alcohol, decyl alcohol, and lauryl alcohol.

B. Polymers of Ethylene Oxide and Propylene Oxide

The compositions of the present invention may comprise from about 0.1% to about 10%, more preferably from about 0.2% to about 5%, and most preferably from about 0.5% to about 3% of a polymer selected from the group consisting of ethylene oxide, propylene oxide, and mixtures thereof. The polymers of the present invention are characterized by the general formula:

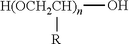

wherein n has an average value of from about 2,000 to about 14,000, preferably from about 5,000 to about 9,000, more preferably from about 6,000 to about 8,000; and wherein R is selected from the group consisting of H, methyl, and mixtures thereof.

When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist.

Polyethylene glycol polymers useful herein that are especially preferred are PEG-2M wherein R equals H and n has an average value of about 2,000 (PEG 2-M is also known as Polyox WSR® N-10 from Union Carbide and as PEG-2,000); PEG-5M wherein R equals H and n has an average value of about 5,000 (PEG 5-M is also known as Polyox WSR® N-35 and Polyox WSR® N-80, both from Union Carbide and as PEG-5,000 and Polyethylene Glycol 300,000); PEG-7M wherein R equals H and n has an average value of about 7,000 (PEG 7-M is also known as Polyox WSR® N-750 from Union Carbide); PEG-9M wherein R equals H and n has an average value of about 9,000 (PEG 9-M is also known as Polyox WSR® N-3333 from Union Carbide); and PEG-14 M wherein R equals H and n has an average value of about 14,000 (PEG 14-M is also known as Polyox WSR® N-3000 from Union Carbide).

C. Water

The compositions of the present invention may comprise from about 50% to about 99.8%, by weight, water. The water phase can optionally include other liquid, water-miscible or water-soluble solvents such as lower alkyl alcohols, e.g. C1-C5 alkyl monohydric alcohols, preferably C2-C3 alkyl alcohols. However, the liquid fatty alcohol must be miscible in the aqueous phase of the composition. Said fatty alcohol can be naturally miscible in the aqueous phase or can be made miscible through the use of co-solvents or surfactants.

D. Conditioning Benefit-Delivery Agent

The compositions of the present invention may comprise from 0% to about 20%, by weight, of one or more conditioning benefit-delivery agents selected from the group consisting of cationic surfactants, cationic polymers, nonvolatile silicones, nonvolatile hydrocarbons, saturated C14 to C22 straight chain fatty alcohols, nonvolatile hydrocarbon esters, and mixtures thereof.

Cationic surfactants useful in compositions of the present invention contain amino or quaternary ammonium moieties. The cationic surfactant is preferably, though not necessarily, insoluble in the compositions hereof. Cationic surfactants are preferably utilized at levels of from about 0.1% to about 10%, more preferably from about 0.25% to about 5%, most preferably from about 0.5% to about 2%, by weight of the composition. Cationic surfactants among those useful herein are disclosed in the following documents: McCutcheon's Publications, McCutcheon's Emulsifiers & Detergents (North American Edition 2008); Schwartz, et al., Surface Active Agents: Their Chemistry and Technology, Interscience Publishers, 1949; U.S. Pat. No. 3,155,591; U.S. Pat. No. 3,929,678; U.S. Pat. No. 3,959,461; and U.S. Pat. No. 4,387,090.

Cationic polymers useful in compositions of the present invention are preferably water soluble, but not necessarily soluble in the composition. Cationic polymers are typically used in the same ranges as disclosed above for cationic surfactants. Suitable cationic hair conditioning polymers include, for example: copolymers of 1-vinyl-2-pyrrolidone and 1-vinyl-3-methyl-imidazolium salt (e.g., chloride salt) (referred to in the industry by the Cosmetic, Toiletry, and Fragrance Association, “CTFA,” as Polyquaternium-16), such as those commercially available from BASF Wyandotte Corp. (Parsippany, N.J., USA) as LUVIQUAT (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) as GAFQUAT (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from 3 to 5 carbon atoms, as described in U.S. Pat. No. 4,009,256. Other useful cationic polymers include polysaccharide polymers, such as cationic cellulose derivatives and cationic starch derivatives; cationic guar gum derivatives, such as guar hydroxypropyltrimonium chloride (commercially available from Celanese Corp. in their Jaguar R series); quaternary nitrogen-containing cellulose ethers (e.g., U.S. Pat. No. 3,962,418); and copolymers of etherified cellulose and starch (e.g., U.S. Pat. No. 3,958,581).

Nonvolatile silicones useful in compositions of the present invention may be soluble or insoluble. By soluble what is meant is that the silicone conditioning active is miscible with the aqueous carrier of the composition so as to form part of the same phase. By insoluble what is meant is that the silicone forms a separate, discontinuous phase from the aqueous carrier, such as in the form of an emulsion or a suspension of droplets of the silicone. The nonvolatile silicone will be used in the compositions hereof at levels of from about 0.05% to about 10% by weight of the composition, preferably from about 0.1% to about 8%. In one embodiment, the nonvolatile silicone is insoluble and has a viscosity of from about 1,000 to about 2,000,000 centistokes at 25° C., more preferably from about 10,000 to about 1,800,000, even more preferably from about 100,000 to about 1,500,000. The viscosity can be measured by means of a glass capillary viscometer as set forth in Dow Corning Corporate Test Method CTM0004, Jul. 20, 1970. Exemplary insoluble, nonvolatile silicones include polyalkyl siloxanes, polyaryl siloxanes, polyalkylaryl siloxanes, polyether siloxane copolymers, and mixtures thereof. An optional ingredient that can be included in the nonvolatile silicone is silicone resin. Silicone resins can enhance deposition of silicone on the hair and can enhance the glossiness of hair with high refractive index volumes. Background material on silicones including sections discussing silicone fluids, gums, and resins, as well as manufacture of silicones, can be found in Encyclopedia of Polymer Science and Engineering, Volume 15, Second Edition, pp 204-308, John Wiley & Sons, Inc., 1989.

E. Additional Benefit-Delivery Agents

In particular embodiments, the composition of the present invention may comprise one or more of the hair-care or skin-care benefit-delivery agents described below. Exemplary hair-care benefit-delivery agents include, but are not limited to: anti-dandruff actives (e.g., pyridinethione salts; zinc carbonate; tea tree oil; azoles, such as ketoconazole, econazole, and elubiol; selenium sulfide; particulate sulfur; keratolytic actives; and mixtures thereof); other conditioning actives; hair-hold polymers; detersive surfactants such as anionic, nonionic, amphoteric, and zwitterionic surfactants; additional thickening actives and suspending actives such as xanthan gum, guar gum, hydroxyethyl cellulose, methyl cellulose, hydroxyethylcellulose, starch and starch derivatives; viscosity modifiers such as methanolamides of long chain fatty acids such as cocomonoethanol amide; crystalline suspending actives; pearlescent aids such as ethylene glycol distearate; preservatives such as benzyl alcohol, methyl paraben, propyl paraben and imidazolidinyl urea; polyvinyl alcohol; ethyl alcohol; pH adjusting actives, such as citric acid, sodium citrate, succinic acid, phosphoric acid, sodium hydroxide, sodium carbonate; salts, in general, such as potassium acetate and sodium chloride; coloring actives, such as any of the FD&C or D&C dyes; hair oxidizing (bleaching) actives, such as hydrogen peroxide, perborate and persulfate salts; hair reducing actives, such as the thioglycolates; perfumes; sequestering actives, such as disodium ethylenediamine tetra-acetate; and polymer plasticizing actives, such as glycerin, disobutyl adipate, butyl stearate, sunscreens, and propylene glycol.

Exemplary skin-care benefit-delivery agents include, but are not limited to: sugar amines, peptides, sunscreens, retinoids, oil control agents, N-acyl amino acid compounds, tanning actives, anti-acne actives, desquamation actives, anti-cellulite actives, chelating agents, skin lightening agents, flavonoids, protease inhibitors, tyrosinase inhibitors, non-vitamin antioxidants and radical scavengers, preservatives, depilatories, hair growth regulators, anti-wrinkle actives, anti-atrophy actives, minerals, phytosterols and/or plant hormones, anti-inflammatory agents, antimicrobials, and antifungals. Further suitable benefit-delivery agents include caffeine; tea extracts, e.g. white tea extract and green tea extract; ginseng; cucumber extract; rosehip oil; date palm kernel extract; witch hazel extract; dill extract; tetrahydrocurcmin; turmerone; and other natural or botanical compounds. Many of these benefit-delivery agents are provided in further detail in U.S. Patent Application Publication Nos. US 2006/0275237A1, US 2004/0175347A1, and US 2006/0263309A1; and the Cosmetic, Toiletry, and Fragrance Association International Cosmetic Ingredient Dictionary and Handbook, 11^{th} ed.

The compositions of the present invention may also contain vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts. Such benefit-delivery agents are generally used individually at levels from about 0.01% to about 10.0%, preferably from about 0.05% to about 5.0% by weight of the composition.

### II. FRAGRANCE DELIVERY

In one aspect, the present invention relates to a method of delivering a fragrance by the use of a moist-heat wrap comprising a suppressed fragrance. In various embodiments, the moist-heat wrap of the present invention may comprise a neat fragrance, a suppressed fragrance, or both. In some embodiments, a consumer may initially perceive a neat fragrance associated with (e.g., in or on) the moist-heat wrap. In other embodiments, a consumer may not initially perceive the neat fragrance. In either case, the consumer may not initially perceive (or only minimally perceive) a suppressed fragrance associated with the moist-heat wrap. Rather, the consumer may later perceive the suppressed fragrance—for example, after a certain amount of time. In one embodiment, a consumer may experience a lavender neat fragrance when removing a moist-heat wrap from its packaging and/or applying the wrap, but may not experience a suppressed ocean fragrance until it is activated ten or twenty minutes later. A suppressing agent may be releaseably associated with a fragrance, thereby suppressing the fragrance from consumer detection until the fragrance is released upon activation. Wet hair or steam (moist heat) may activate the release of the suppressed fragrance, thus enabling it to be perceived by a consumer.

A. Suppressed Fragrances

Moist-heat wraps of the present invention may employ a suppressed fragrance. This type of fragrance utilizes a suppressing agent associated with the perfume molecules to inhibit its expression until activated by one or more mechanisms, such as contact with water or heat generated by the moist-heat wrap. In various embodiments, the suppressed fragrance is released between about 1 minute and about 20 minutes, or at about 60 minutes, or between about 20 minutes and 40 minutes, or at about 30 minutes, or subsequent to moisture generation.

A suppressing agent may be employed in the moist-heat wrap, wherein the suppressing agent comprises a means of attaching to the fragrance to prohibit the scent from being immediately perceived by a consumer. Suppressing agents may limit natural diffusion of and/or immobilize fragrances. This encourages stability of fragrances in a given zone. Exemplary fragrance suppressing agents include, but are not limited to, starches, oligosaccharides (e.g., cyclodextrins), polyethylenes, polyamides, polystyrenes, polyisoprenes, polycarbonates, polyesters, polyacrylates, vinyl polymers, silicas, aluminosilicates, and combinations thereof. In one embodiment, the fragrance suppressing agent is β-cyclodextrin, available from Wacker Chemical Corp. Commercially available encapsulating materials N-Lok™, manufactured by National Starch, Narlex™ (ST and ST2), and Capsul E™ are useful for the present invention. These materials comprise pregelatinized waxy maize starch and optionally, glucose. The starch may be modified by adding monofunctional substituted groups such as octenyl succinic acid anhydride. See U.S. Pat. No. 5,733,272 for examples of preparing cyclodextrin encapsulated perfumes.

Moist-heat wraps of the present invention may employ at least one suppressed fragrance. A representative, non-limiting, list of fragrance materials that may be suppressed with the suppressing agent includes aldehyde, alpha-damascone, alpha-methyl ionone, alpha-pinene, alpha-terpineol, amyl acetate, anethole, benzaldehyde, benzyl acetate, benzyl alcohol, benzyl formate, benzyl propionate, beta-damascone, beta-pinene, beta-terpineol, camphene, camphor, cis-3-hexenyl acetate, cis-citral (neral), citral, citronellal, citronellol, citronellyl acetate, clove oil, decanal, decyl aldehyde, dihydrocitronellal, dihydrolinalool, dihydromyrcenol, dimethyl benzyl carbinyl acetate, dimethyl phenyl carbinol, d-limonene, eucalyptol, eucalyptus, eucalyptus oil, eugenol, gamma-methyl ionone, gamma-terpinene, geranial, geraniol, geranyl acetate, geranyl nitrile, helional, iso-bornyl acetate, iso-menthone, lavender, laevo-menthyl acetate, lemon oil, linalool, linalool oxide, methyl nonyl acetaldehyde, methyl phenyl carbinyl acetate, menthol, menthyl acetate, menthyl lactate, methyl benzyl carbinyl acetate, menthone, myrcene, myrcenyl acetate, myrcenol, nerol, neryl acetate, nonyl acetate, orange oil, paracymene, phenyl ethyl alcohol, phenyl acetaldehyde, tetra-hydro linalool, terpineol, terpinyl acetate, undecalactone, undecylenic aldehyde, vertenex (para-tertiary-butyl cyclohexyl acetate), witch hazel distillate, and combinations thereof. Suppressed fragrances useful herein may also comprise fragrances such as those found in commercially available fine fragrances, colognes, perfumes, and the like—for example, an Escada™, Gucci™, or Hugo Boss™ scent. It will be recognized that additional perfumes/aromatic materials known to a person of ordinary skill in the art of creating fragrances may be used as a suppressed fragrance.

B. Neat Fragrances

Moist-heat wraps of the present invention may additionally or alternatively comprise a neat fragrance comprising any perfume or perfume chemical. In a preferred embodiment, moist-heat wraps comprise neat fragrances to provide the wrap with a desired perfume or an unscented/neutral aroma. In one embodiment, a neat fragrance may be useful to help cover or mask malodors, for example, those resulting from certain chemicals present in the wrap. In another embodiment, a neat fragrance may be used to provide the user with a relaxing or spa-like experience.

The concentration of the neat fragrance in the moist-heat wrap should be effective to provide the desired aroma. As used herein, “unscented” refers to the level of fragrance wherein the level of fragrance is below 0.05% such that the fragrance is absent or undetected. Generally, the concentration of the scented neat fragrance is from at least about 0.05%, from about 0.1%, from about 0.5% but no more than about 20%, no more than about 10%, no more than about 5%, or no more than about 2%, by weight of the wrap.

The neat fragrance may include perfumes/aromatic materials known to a person of ordinary skill in the art of creating fragrances. Typical fragrances are described in Arctander, Perfume and Flavour Chemicals (Aroma Chemicals) Vol. I and II (1969), and Arctander, Perfume and Flavour Materials of Natural Origin (1960). U.S. Pat. No. 4,322,308 and U.S. Pat. No. 4,304,679 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

### III. MOIST-HEAT WRAP

In one embodiment, the moist-heat wrap may comprise a moist substrate, such as a damp towel, which is heated by an outside heating mechanism, such as a hair dryer. In another embodiment, the moist-heat wrap itself may comprise a wet or dry substrate comprising a steam-generating unit. The moist-heat wrap can be a single-use disposable system or it can be incorporated into a reusable or partially-reusable system.

A. Substrate

The moist-heat wrap of the present invention comprises a skin- or hair-facing side and an opposing side. In certain embodiments, the skin- or hair-facing side of the moist-heat wrap may comprise at least one contact surface. The contact surface may be moisture-permeable. Non-limiting examples of suitable materials for the contact surface include fabric, nonwoven fabric, paper, synthetic paper, nylon, vinylon, polyester, rayon, acetate, acrylic, polyethylene, polypropylene, polyvinyl chloride, pulp, cotton, flax, silk, animal hair, gas impermeable films or sheets (e.g., polyvinylidene chloride, polyurethane, polystyrene, rubber, ethylene-vinyl acetate copolymer, etc.) with micropores, cellulose ester, polyvinyl derivatives, polyolefin, polyamide, cellulose, wool, jute, hemp, linen, sisal, ramie, and combinations thereof. In one embodiment, the contact surface is in the form of a sheet and has a moisture penetration by ASTM methods of 4,000 g/m²·24 h or higher, or 8,000 g/m²—24 h or higher. In some embodiments, the contact surface comprises fasteners—for example, adhesive strips, buttons, or Velcro—to secure the moist-heat wrap to the skin or hair.

In certain embodiments, the side opposing the skin- or hair-facing side of the moist-heat wrap may comprise at least one non-contact surface. The non-contact surface may be moisture-impermeable and may direct steam towards the contact surface of the moist-heat wrap. Non-limiting examples of suitable materials for the non-contact surface include nonwovens, paper-foil laminate, foil, foamed plastics (e.g., polyethylene, polypropylene, polystyrene, polyester, and polyurethane-based foams), foamed-plastic sheet, plastic film, sponge, glass wool, fiberglass, and combinations thereof.

In particular embodiments, the moist-heat wrap may comprise a temperature-regulating material. The thickness of the temperature-regulating material, each of the structural element materials forming the regulating material, their combined state when several structural elements are stacked, the stacking method, etc. may be determined so that a specific amount of steam at a specific temperature may be obtained. Non-limiting examples of suitable materials for the temperature-regulating material include fabric, nonwoven fabric, paper, synthetic paper, porous films or porous sheets made from plastic or rubber, foamed plastics (e.g. urethane foam) with through holes, metal foils with through holes, and the like.

In some embodiments, the moist-heat wrap may comprise a dye, colorant, pigment, or the like which gives the moist-heat wrap a hue. In other embodiments, the moist-heat wrap may comprise a solid hue or a pattern of various hues. Different hues or patterns may signal to a consumer that the wrap has a certain fragrance, is multi-fragranced, has a fragrance shift, or has a fragrance which changes in intensity. A hue or pattern may be selected to represent a neat fragrance to enhance consumer delight. For example, if a watery neat fragrance, a blue or green hue may be selected; if a citrus neat fragrance, a yellow, orange, or lime green hue may be selected. In further embodiments, suppressed fragrances and neat fragrances may comprise a dye, colorant, pigment, or the like, such that each fragrance has a different color or hue; many combinations are possible by varying the hues.

B. Steam-Generating Unit

The moist-heat wrap of the present invention comprises a steam-generating unit that is safe for application to skin and/or hair. “Steam,” as used herein, refers to gasified water, water vapor, fine water droplets, moist heat, water in a gaseous state, and the like. Steam is able to swell or moisturize hair and skin to enhance penetration of active ingredients into and/or enhance the distribution of materials onto the hair or skin—one example is distributing silicone onto the hair surface to reduce friction. These benefits can only be achieved if a moist-heat unit delivers a particular, effective amount of steam. In various embodiments, the amount of steam released from the steam-generating unit and available to the skin or hair may be 0.01 mg/cm²·min or greater, or 0.5 mg/cm²·min or greater. The amount of steam released, SR (in mg/cm²·min), may be calculated by the following formula: SR=1000(Wt₀−Wt₁₅)/15SA; wherein Wt₀ (g)=weight at time evaluation begins, Wt₁₅=weight after 15 minutes, and SA (cm²)=surface area of the part where the surface of the steam generating unit is placed directly on a top-pan scale capable of measuring up to 1 mg units under atmosphere in a room temperature environment (20° C., 65% rel. humid.) and weight is determined 15 minutes later. In order to deliver a particular, effective amount of steam, the contact surface of the steam-generating unit is, in one embodiment, in the form of a moisture-permeable sheet.

In one embodiment, the steam-generating unit may employ a heating part which undergoes an exothermic reaction to generate steam, thus eliminating the need for a separate vaporizing part. An example of this is a steam-generating composition that comprises metal powder, salt, and water, and releases steam with oxidation of the metal powder. In one embodiment, iron powder goes through an exothermic reaction as shown by the following formula to release the water within the system as steam: Fe+(¾)O₂+(3/2)H₂O→Fe(OH)₃+96 kcal. This type of exothermic reaction by metal powder is commonly used in a chemical pocket heater, which isn't generally made from a sheet that is moisture permeable. The exothermic reaction of the present invention, however, is meant to generate steam. Accordingly, the sheet is preferably moisture permeable, unlike traditional chemical pocket heaters. See U.S. Pat. No. 6,629,964 and Japanese Patent Laid-Open No. 62-172907 for further details.

In other embodiments, the steam-generating unit may employ chemical energy to generate steam. In one embodiment, the unit may employ heat of hydration of inorganic salts or energy produced by neutralization of acids and bases to generate steam. For instance, the steam-generating unit may comprise a heating part which generates heat and a vaporizing part which generates steam. The heating part may heat via heat of neutralization or heat of hydration. The vaporizing part may comprise a fabric, a nonwoven fabric, a fiber aggregate of paper, or a porous material comprising water. In further embodiments, to generate steam, the unit may employ electrical energy, reheatable gels, or other devices known to one of skill in the art.

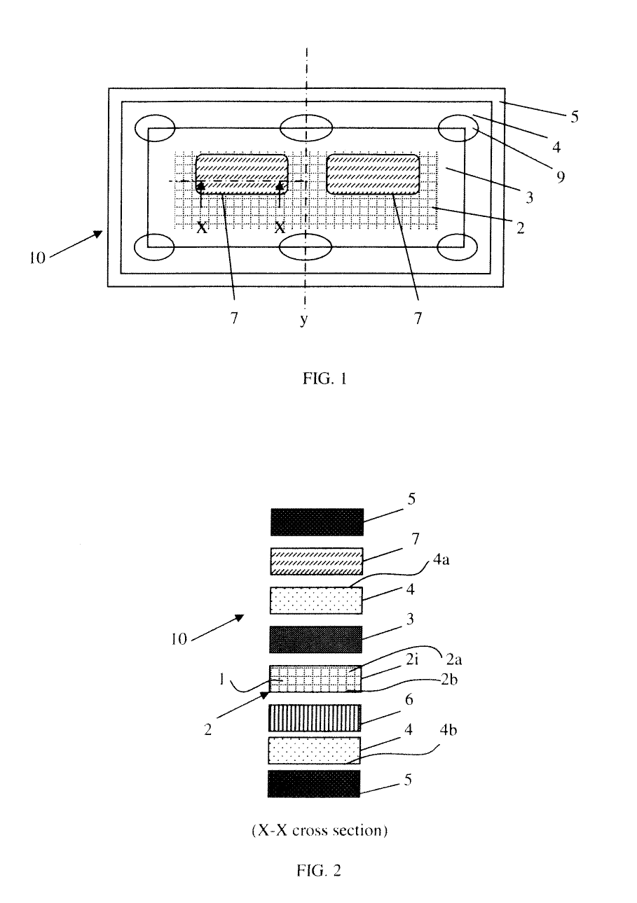

A preferred mode of the present invention is described below in detail while referring to the figures. The same symbols represent the same or identical structural elements in each of the figures. FIG. 1 is a top view and FIG. 2 is a x-x cross section of moist-heat wrap 10 of one mode of the present invention where an optional temperature-regulating material has been placed in between the steam-generating unit and the surface of the moist-heat wrap in order to keep the temperature of the steam released from the surface of the moist-heat wrap at 50° C. or lower. The moist-heat wrap 10 comprises a steam-generating unit 2 where a steam-generating composition 1 is held inside a moisture-permeable inner bag 2i, a temperature-regulating material 3 piled on the steam-generating unit 2, a moisture-permeable outer bag 4 holding all of these, and further outside of these, an optional active layer 7, and a package 5. The moist-heat wrap 10 is used by opening the package 5 (e.g., tearing a sealed bag open) at the time of use, taking out its contents, and applying the moisture-permeable outer bag 4 so that the contact surface 4a (or optional active layer 7) contacts the hair or skin and the non-contact surface 4b is facing away from the hair or skin.

In one embodiment, the steam-generating composition 1 held inside the moisture-permeable inner bag 2i is made from a composition containing the same metal powder (e.g., iron, aluminum, zinc, copper), salts (e.g., sodium chloride, potassium chloride, calcium chloride, magnesium chloride), and water as the exothermic composition used for conventional chemical pocket heaters by conventional methods and generates steam as a result of the oxidation reaction of the metal powder. Of these, iron powder is preferred as the metal powder in terms of economics, reactivity, and safety. Moreover, the steam-generating composition 1 can also contain a variety of components, such as moisture-retaining agents (e.g., vermiculite, calcium silicate, silica gel, silica porous substances, alumina, pulp, wood powder, water-absorbing polymers) and reaction promoters (e.g., activated carbon, carbon black, graphite), etc. The amount of steam-generating composition 1 held inside the moisture-permeable inner bag 2i is determined as needed in accordance with the desired amount of steam release and the steam temperature.

The moisture-permeable inner bag 2i comprises a moisture-permeable side 2a and a moisture-impermeable side 2b to direct the steam towards the contact surface 4a. In some embodiments, the moisture-permeable inner bag 2i may comprise two moisture-permeable sides 2a and 2b; in these instances, a moisture-impermeable sheet 6, for instance, a piled sheet of ethylene-vinyl acetate copolymer and polyethylene, may be piled on the surface 2b. Thus, dispersing of the steam that has been released from the steam-generating composition 1 is prevented by using this moisture-impermeable sheet 6 (or moisture-impermeable side 2b of the inner bag 2i) and the steam can pass through the moisture-permeable side 2a of the inner bag 2i and thus be more effectively guided toward the body. In particular embodiments, instead of comprising temperature-regulating material 3, the moisture-permeable inner bag 2i may have additional function similar to the temperature regulating material 3. In some embodiments, the contact surface 4a comprises fasteners 9—for example, adhesive, buttons, clips, ties, or Velcro—to secure the moist-heat wrap to the skin or hair.

### IV. METHODS

The present invention further relates to a method of providing benefits to the hair. In one embodiment, the method comprises the steps of: (a) applying a hair-care composition to the hair; and (b) applying a moist-heat wrap to the hair, wherein the moist-heat wrap comprises a suppressed fragrance releaseably associated with a suppressing agent. The benefit may be selected from the group consisting of hair conditioning, hair shine, smooth hair, soft hair, healthy hair, hair strand alignment, dandruff-free hair, hair coloring, and combinations thereof.

The hair-care compositions of the present invention are used in conventional ways to provide the conditioning and other benefits of the present invention. The use of the composition depends upon the type of composition employed but generally involves application of an effective amount of the product to the hair, which may then be rinsed from the hair (as in the case of hair rinses) or allowed to remain on the hair (as in the case of gels, lotions, and creams). “Effective amount” means an amount sufficient enough to provide a dry combing benefit. In general, from about 1 g to about 50 g is applied to the hair on the scalp. The composition is distributed throughout the hair, typically by rubbing or massaging the hair and scalp. In some embodiments, the composition is applied to wet or damp hair prior to drying of the hair. In other embodiments, the composition is applied to dry hair.

One or more hair-care compositions may be applied prior to, simultaneously with, as a part of, or subsequent to application of the moist-heat wrap. In one embodiment i) a hair-care composition is applied to the hair, ii) rinsed off, then iii) a second hair-care composition is applied to the hair, and iv) a moist-heat wrap is applied to the hair. In another embodiment i) a hair-care composition is applied to the hair, ii) a moist-heat wrap is applied to the hair. In another embodiment i) one hair-care composition is applied to the hair, ii) a moist-heat wrap is applied to the hair, and iii) a second hair-care composition is applied to the hair after the moist-heat wrap is removed from the hair. The hair-care composition can provide a variety of benefits including, but not limited to, conditioning, shine enhancement, healthier hair, healthier scalp, anti-dandruff, and/or any combination thereof. The method can further comprise the step of increasing hair moisture level by at least about 300% versus hair moisture level prior to application of the system, over a time period of less than about 30 minutes. In various embodiments, the moist-heat wrap is left on the hair for about 1 minute to about 20 minutes, or for about 60 minutes, or for about 20 minutes to 40 minutes, or for about 30 minutes.

### V. EXAMPLES

The following further describe and demonstrate embodiments within the scope of the present invention. The examples are given solely for the purpose of illustration and are not to be construed as limitations of the present invention, as many variations thereof are possible without departing from the spirit and scope of the invention.

A. Suppressed Fragrance

To encapsulate a volatile perfume oil, first, choose a perfume with small enough molecules that will easily enter the pores of β-cyclodextrin—for example, an essential oil containing lemon components. Then, with a DAK high speed mixer, slurry the β-cyclodextrin with the perfume at a 10:1 ratio. Cover and let stand overnight to load as much perfume as possible into the β-cyclodextrin. Next, mix the β-cyclodextrin with water to a slurry and air-dry. The white brittle solid obtained should have no (or very little) odor at all. If it does, wash the excess perfume off by stirring with hexane and filtering. Let air dry and grind to a fine powder. See U.S. Pat. No. 5,733,272 for examples of preparing cyclodextrin-encapsulated perfumes that use moisture to release perfumes. Use a brush or pipette to brush or streak the solid onto the desired moist-heat wrap. Let air dry.

B. Hair-Care Compositions

1. Rinse-Off Formulation Examples:

| Ingredients | Ex. 1 | Ex. 2 | Ex. 3 | Ex. 4 | Ex. 5 | Ex. 6 |
|---|---|---|---|---|---|---|
| Stearamidopropyldimethylamine | 2.0 | | | | | |
| (SAPDMA)¹ | | | | | | |
| Behenamidopropyl | | 2.3 | 2.3 | 2.3 | | |
| Dimethylamine² | | | | | | |
| Behenyl trimethyl ammonium | | | | | 2.8 | 2.8 |
| chloride³ | | | | | | |
| L-Glutamic Acid⁴ | .64 | .64 | .64 | .64 | | |
| Cetyl Alcohol⁵ | 2.5 | 2.5 | 2.5 | 2.5 | 1.9 | 1.9 |
| Stearyl Alcohol⁶ | 4.5 | 4.5 | 4.5 | 4.5 | 4.6 | 4.6 |
| Amodimethicone⁷ | | 2.0 | | | 2.0 | |
| Amodimethicone⁸ | | | 3.0 | | | 3.0 |
| Dimethicone/Cyclomethicone⁹ | 4.2 | | | | | |
| Dimethicone blend¹⁰ | | | | 4.2 | | |
| Dimthicone¹¹ | | 3.0 | | | 3.0 | |
| Ethylene Diamine Tetraacetic | .1 | | | | | |
| Acid (EDTA) | | | | | | |
| Disodium EDTA | | .127 | .127 | .127 | .127 | .127 |
| Preservative | .40005 | .40005 | .40005 | .40005 | .40005 | .40005 |
| Perfume | .25 | .5 | .5 | .5 | .5 | .5 |
| Panthenyl ethyl ether | .05 | .05 | | .05 | .05 | |
| Panthenol | .05 | .05 | | .05 | .05 | |
| Sodium hydroxide (NaOH) | | .014 | .014 | .014 | .014 | .014 |
| Water | q.s. | q.s. | q.s. | q.s. | q.s. | q.s. |

¹Stearamidopropyl dimethylamine: Lexamine S-13 available from Inolex
²Behenamidopropyl Dimethylamine (IncromineBB) available from Croda
³Behenyl trimethyl ammonium chloride/Isopropyl alcohol: Genamin KDMP available from Clariant
⁴Glutamic acid: available from Ajinomoto
⁵Cetyl alcohol: Konol series available from Shin Nihon Rika
⁶Stearyl alcohol: Konol series available from Shin Nihon Rika
⁷Terminal aminosilicone, AP type, available from Momentive; viscosity range from 220,000-245,000 cs
⁸Terminal aminosilicone, AP type, available from Momentive; viscosity 10,000 cs
⁹Dimethicone/cyclomethicone: a blend of dimethicone having a viscosity of 500,000 mPa * s and cyclopentasiloxane available from Momentive
¹⁰Dimethicone blend: a blend of dimethicone having a viscosity of 500,000 mPa * s and dimethicone having a viscosity of 200 mPa * s available from Momentive
¹¹Dow Corning 200 Fluid, 10 cs

Compositions in accordance with above examples can be prepared by any conventional method well known in the art. They are suitably made as follows: deionized water is heated to 85° C. and cationic surfactants and high melting point fatty compounds are mixed in. If necessary, cationic surfactants and fatty alcohols can be pre-melted at 85° C. before addition to the water. The water is maintained at a temperature of about 85° C. until the components are homogenized, and no solids are observed. The mixture is then cooled to about 55° C. and maintained at this temperature, to form a gel matrix. Aminosilicones, or a blend of aminosilicones and a low viscosity fluid, or an aqueous dispersion of an aminosilicione, or the dimethicone, are added to the gel matrix. When included, other additional components such as perfumes and preservatives are added with agitation. The gel matrix is maintained at about 50° C. during this time with constant stiffing to assure homogenization. After it is homogenized, it is cooled to room temperature.

2. Leave-on Formulation Examples:

| Ingredients | Ex. 1 | Ex. 2 |
|---|---|---|
| Quaternium-18 | 0.45 | 0.45 |
| Cetearyl Alcohol and | 0.30 | 0.30 |
| Polysorbate-60 | | |
| Glyceryl Stearate | 0.15 | 0.15 |
| Cetyl Alcohol | 0.81 | 0.81 |
| Stearyl Alcohol | 0.54 | 0.54 |
| Stearamidopropyl | 0.60 | 0.60 |
| Dimethylamine | | |
| Oleyl Alcohol | 0.15 | 0.15 |
| PEG-2M | 0.30 | 0.30 |
| Hydroxyethylcellulose | 0.15 | 0.15 |
| Dimethicone | 6.0 | 2.0 |
| Citric Acid | 0.10 | 0.10 |
| EDTA | 0.05 | 0.05 |
| Preservative | 0.76 | 0.76 |
| Fragrance/Perfume | 0.40 | 0.40 |
| Panthenyl Ethyl Ether | 0.05 | 0.05 |
| Panthenol | 0.09 | 0.09 |
| Water | q.s. | q.s. |

Compositions in accordance with above examples can be prepared by any conventional method well known in the art. They are suitably made as follows: deionized water is heated to 85° C. and the first nine ingredients are mixed in. The water is maintained at a temperature of about 85° C. until the components are homogenized, and no solids are observed. The mixture is then cooled to about 55° C. The remaining materials are mixed in. The gel matrix is maintained at about 50° C. during this time with constant stirring to assure homogenization. After it is homogenized, it is cooled to room temperature.

C. Steam-Generating Unit

A moist-heat wrap as shown in FIGS. 1 and 2 may be made as follows. First, mix 5 g of a mixture of 1 part by weight water-absorbing polymer (Nihon Shokubai Co., Ltd., brand name: Aqualik CA), 3 parts by weight silica gel (Wako Junyaku Co., Ltd., brand name: Wakogel C-200), and 10 parts by weight of aqueous 12.5 wt % sodium chloride solution and 10 g iron powder (Dowa Teppun Kogyo Co., Ltd., brand name: RKH) to obtain a steam-generating composition 1. Next, pack 3 g of the steam-generating composition 1 into a 3×3 cm² small square bag comprising a vinyl-coated sheet on one side (Nitto Denko Co., Ltd., brand name: Nitotack) and a sheet of moisture-permeable nonwoven (Mitsui Kagaku, brand name: Syntex MB, net: 15 g/m²) on the other side. Then, place the small bag on the nonwoven and anchor the bottom surface of the bag to the nonwoven (Chisso Co., Ltd., brand name: Air-raid, net: 24 g/m²) support with the surface made from moisture-permeable nonwoven facing up. The temperature regulating material 3 shown in FIG. 2 may have a piled structure. Pile, in order, one layer of paper (Kreshia, brand name: Kimtowel), two layers of nonwoven (Chisso, brand name: Air-raid, net: 24 g/m²), one layer of paper (Kreshia, brand name: Kimtowel), and two layers of nonwoven (Chisso, brand name: Air-raid, net: 24 g/m²) and place the entire unit of temperature regulating material 3 in the moisture-permeable outer bag 4 comprising moisture-permeable nonwoven (Mitsui Kagaku, brand name: Syntex MB, net: 15 g/m²). Then, seal the entire outside by an air-tight bag (sealed bag) 5 (Asahi Kasei Polyplex Co., Ltd., brand name: Hiryu) to obtain the moist-heat wrap 10. The moist-heat wrap obtained may be taken out of the air-tight bag 5 and used.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of providing a benefit to the hair comprising:
(a) applying a hair-care composition to the hair; and
(b) applying a moist-heat wrap to the hair, wherein the moist-heat wrap comprises a suppressed fragrance releaseably associated with a suppressing agent;
wherein the benefit is selected from the group consisting of hair conditioning, hair shine, smooth hair, soft hair, healthy hair, hair strand alignment, dandruff-free hair, hair coloring, and combinations thereof.

2. The method of claim 1, wherein the suppressed fragrance is selected from the group consisting of lemon oil, lavender, fine fragrances, and combinations thereof.

3. The method of claim 1, wherein the suppressed fragrance is lemon oil.

4. The method of claim 1, wherein the suppressing agent is selected from the group consisting of starches, oligosaccharides, polyethylenes, polyamides, polystyrenes, polyisoprenes, polycarbonates, polyesters, polyacrylates, vinyl polymers, silicas, aluminosilicates, and combinations thereof.

5. The method of claim 1, wherein the suppressing agent is cyclodextrin.

6. The method of claim 1, wherein the suppressing agent is β-cyclodextrin.

7. The method of claim 1, wherein the hair-care composition is selected from the group consisting of a leave-on conditioner, a rinse-off conditioner, and combinations thereof.

8. The method of claim 1, wherein the moist-heat wrap further comprises a contact side and a non-contact side, and wherein the location of the suppressed fragrance is selected from the group consisting of the contact side, the non-contact side, or the entire moist-heat wrap.

9. The method of claim 1, wherein the moist-heat wrap further comprises a neat fragrance.

10. The method of claim 9, wherein the neat fragrance and the suppressed fragrance are different, wherein said difference is selected from the group consisting of: same chemical make-ups but different concentrations, same concentrations but different chemical make-ups, different chemical make-ups and different concentrations, and combinations thereof.

11. The method of claim 1, wherein the hair-care composition comprises a neat fragrance.

12. The method of claim 1, wherein the hair-care composition further comprises a suppressed fragrance releaseably associated with a suppressing agent.

13. The method of claim 1, wherein two hair-care compositions are applied to the hair.

14. The method of claim 1, wherein the composition comprises:
(a) from about 0.1% to about 10%, by weight of a monohydric, fatty alcohol having a melting point of 30° C. or lower, the fatty alcohol selected from the group consisting of unsaturated straight chain fatty alcohols, saturated branched chain fatty alcohols, saturated C8-C12 straight chain fatty alcohols, and mixtures thereof;
(b) from about 0.1% to about 10%, by weight, of a polymer selected from the group consisting of ethylene oxide, propylene oxide, and mixtures thereof, having the general formula: 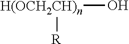
wherein n has an average value of from about 2,000 to about 14,000, and wherein R is selected from the group consisting of H, methyl, and mixtures thereof;
(c) from about 50% to about 99.8%, by weight, water; and
(d) from 0% to about 20%, by weight, of one or more conditioning actives selected from the group consisting of cationic surfactants, cationic polymers, nonvolatile silicones, nonvolatile hydrocarbons, saturated C14 to C22 straight chain fatty alcohols, nonvolatile hydrocarbon esters, and mixtures thereof.

15. The method of claim 1, wherein the composition is applied to wet hair.

16. The method of claim 1, wherein the composition is applied to dry hair.

17. The method of claim 1, wherein the moist-heat wrap is left on the hair for about 20 to about 40 minutes.

18. The method of claim 1, wherein the suppressed fragrance disassociates with the suppressing agent about 20 to about 40 minutes after contact with water or heat.

19. The method of claim 1, wherein the moist-heat wrap is left on the hair for about 30 minutes.

20. The method of claim 1, wherein the hair-care composition is applied to the roots of the hair.

21. The method of claim 1, wherein the hair-care composition is applied to the bottom half of the hair.

22. The method of claim 1, wherein the hair-care composition is applied to the ends of the hair.
